(19) United States
(12) Patent Application Publication
(10) Pub. No.: US 2018/0242562 A1
(43) Pub. Date: Aug. 30, 2018

(54) BIRDS, METHOD FOR PRODUCING BIRDS, AND BIRD EGGS

(71) Applicants: Hiroshima University, Higashihiroshima-shi, Hiroshima (JP); Kewpie Corporation, Shibuya-ku, Tokyo (JP)

(72) Inventors: Hiroyuki Horiuchi, Higashihiroshima-shi, Hiroshima (JP); Ryo Ezaki, Higashihiroshima-shi, Hiroshima (JP); Takashi Yamamoto, Higashihiroshima-shi, Hiroshima (JP); Tetsushi Sakuma, Higashihiroshima-shi, Hiroshima (JP); Akihiro Handa, Chofu-shi, Tokyo (JP); Ryou Sasahara, Chofu-shi, Tokyo (JP); Daisuke Kodama, Chofu-shi, Tokyo (JP)

(21) Appl. No.: 15/755,455

(22) PCT Filed: Jul. 20, 2016
(86) PCT No.: PCT/JP2016/071237

§ 371 Date: Feb. 26, 2018

(30) Foreign Application Priority Data
Aug. 27, 2015 (JP) 2015-168372

(51) Int. Cl. A01K 67/027; C12N 15/09

(52) U.S. Cl. CPC A01K 67/027; C12N 5/10; C12N 15/09

(57) ABSTRACT

According to this invention, a bird lays an egg that does not contain an artificially introduced foreign gene in genome thereof and has an ovomucoid content lower than that in a wild type thereof. A method for producing a bird includes a modification step of cleaving the ovomucoid gene locus in a pluripotent stem cell of a bird with programmable endonuclease so as to modify the ovomucoid gene locus and a transplantation step of transplanting the pluripotent stem cell in which the ovomucoid gene locus is modified into an embryo of a bird. A bird egg does not contain an artificially introduced foreign gene in genome thereof and has an ovomucoid content lower than that in a wild type thereof.

### TECHNICAL FIELD

The present disclosure relates to a bird, a method for producing a bird and a bird egg.

### BACKGROUND ART

Chicken eggs are the first cause of food allergy for Japanese. Some proteins in chicken eggs function as allergens that induce food allergy. Examples of allergens contained in chicken eggs include ovomucoid, ovalbumin, lysozyme, and ovotransferrin.

In order not to prevent food allergy caused by a chicken egg as a causative food, there have been attempts to remove the above allergens from chicken eggs. Allergens can be removed from chicken eggs by producing chickens, in which genes encoding allergens have been destroyed.

Non Patent Literature 1 discloses a genetically modified chicken, in which the ovalbumin gene is knocked out by transcription activator-like effector nuclease (TALEN). It is considered possible to obtain chicken eggs free of ovalbumin using the genetically modified chickens.

### CITATION LIST

### Non Patent Literature

Non Patent Literature 1: Park T S and four others, “Targeted gene knockout in chickens mediated by TALENs.,” Proceedings of the National Academy of Sciences of the United States of America, 2014, 111(35), 12716-12721

### SUMMARY OF INVENTION

### Technical Problem

Ovalbumin-fee chicken eggs also contain ovomucoid at about 10% in albumen. Ovomucoid is a protein having the strongest allergenicity in chicken eggs. Since ovomucoid is highly physicochemically stable, allergenicity of ovomucoid is maintained even after heating. Therefore, it is difficult to say that allergenicity of chicken eggs has been sufficiently reduced even by, for example, preventing chicken eggs from containing ovalbumin or deactivating ovalbumin by heating according to the method disclosed in Non Patent Literature 1.

In addition, primordial germ cells are used in Non Patent Literature 1. Since the number of primordial germ cells present in the developmental process is very small, there is a demand for primordial germ cell culture technology. Hitherto, methods for culturing a plurality of primordial germ cells have been reported. However, such reports are limited to several research institutes, and it is impossible to carry out culture even in accordance with the reported techniques, which disadvantageously results in poor certainty.

The present disclosure has been made in view of the above-described circumstances. It is an objective of the present disclosure to provide a bird, by which egg allergenicity can be sufficiently reduced, a method for producing such bird, and a bird egg having sufficiently reduced allergenicity.

### Solution to Problem

The inventors of the present disclosure knocked out the chicken ovomucoid gene by a conventional homologous recombination method. However, cell culture is time-consuming in the conventional homologous recombination method, which causes damage on cells and the occurrence of abnormal karyotype. Further, even by crossing chimeric chickens obtained by the homologous recombination method, ovomucoid gene-knockout homozygous chickens could not be obtained. Eggs laid by such chimeric chickens contained recipient genome-derived ovomucoid, and egg allergenicity was not sufficiently reduced.

As a result of intensive studies, the inventors of the present disclosure completed the present disclosure by applying genome editing technology. Specifically, a bird according to the first aspect of the present disclosure lays an egg that does not contain an artificially introduced foreign gene in genome thereof and has an ovomucoid content lower than that in a wild type thereof.

In this case, the bird according to the first aspect of the present disclosure may include a termination codon in at least one of first to third exons from a 5′ end of an ovomucoid gene locus or may not include an initiation codon in a first exon from a 5′ end of an ovomucoid gene locus.

In addition, in the bird according to the first aspect of the present disclosure, a signal sequence within the ovomucoid gene locus may be modified.

In addition, the bird according to the first aspect of the present disclosure may be a chicken.

A method for producing a bird according to the second aspect of the present disclosure comprises:

a modification step of cleaving an ovomucoid gene locus of a pluripotent stem cell of a bird with a programmable endonuclease, thereby modifying the ovomucoid gene locus; and

a transplantation step of transplanting the pluripotent stem cell containing the modified ovomucoid gene locus into an embryo of a bird.

In this case, the programmable endonuclease may be transcription activator-like effector nuclease.

In addition, the transcription activator-like effector nuclease may comprise a first nuclease and a second nuclease, the first nuclease may comprise an amino acid sequence set forth in SEQ ID NO: 4, and the second nuclease may comprise an amino acid sequence set forth in SEQ ID NO: 5.

In addition, the transcription activator-like effector nuclease may comprise a third nuclease and a fourth nuclease, the third nuclease may comprise an amino acid sequence set forth in SEQ ID NO: 6, and the fourth nuclease may comprise an amino acid sequence set forth in SEQ ID NO: 7.

In addition, the programmable endonuclease may be Cas9 nuclease in a Clustered Regularly Interspaced Short Palindromic Repeat-CRISPR associated protein system.

In addition, the Cas9 nuclease may cleave double-strand DNA in a region comprising a base sequence set forth in SEQ ID NO: 8 of the ovomucoid gene locus.

The Cas9 nuclease may cleave double-strand DNA in a region comprising a base sequence set forth in SEQ ID NO: 9 of the ovomucoid gene locus.

The Cas9 nuclease may cleave double-strand DNA in a region comprising a base sequence set forth in SEQ ID NO: 10 of the ovomucoid gene locus.

The Cas9 nuclease may cleave double-strand DNA in a region comprising a base sequence set forth in SEQ ID NO: 11 of the ovomucoid gene locus.

The Cas9 nuclease may cleave double-strand DNA in a region comprising a base sequence set forth in SEQ ID NO: 12 of the ovomucoid gene locus.

The Cas9 nuclease may cleave double-strand DNA in a region comprising base sequence set forth in SEQ ID NO: 13 of the ovomucoid gene locus.

In addition, in the modification step, an ovomucoid gene locus of an epiblast-derived pluripotent stem cell may be modified.

A bird egg according to the third aspect of the present disclosure does not contain an artificially introduced foreign gene in genome thereof and has an ovomucoid content lower than that in a wild type thereof.

### Advantageous Effects of Invention

According to the present disclosure, allergenicity of an egg can be sufficiently reduced. In addition, according to the present disclosure, a bird egg having sufficiently reduced allergenicity can be obtained.

### BRIEF DESCRIPTION OF DRAWINGS

FIG. 1 depicts the base sequence of the first exon (exon 1) from the 5′ end of the chicken ovomucoid gene locus, the base sequence of the second exon (exon 2) therefrom, and the base sequence of the third exon (exon 3) therefrom;

FIG. 2 depicts a TALEN-recognition region identified in exon 1 and that in exon 3;

FIG. 3 depicts relative cleavage activity of TALEN targeting exon 1 and that of TALEN targeting exon 3;

FIG. 4 depicts the base sequence of the target region in the genome of epiblast-derived pluripotent stem cells transfected with a TALEN expression vector targeting exon 3;

FIG. 5 depicts relative cleavage activity of TALEN targeting exon 3 and that of high activity-type TALEN targeting exon 1;

FIG. 6 depicts the configuration of a TALEN expression vector;

FIG. 7 depicts relative cleavage activity of TALEN in chicken cells;

FIG. 8A depicts mutagenesis of epiblast-derived pluripotent stem cells with the use of the TALEN expression vector upon genomic polymerase chain reaction (PCR);

FIG. 8B depicts mutagenesis of epiblast-derived pluripotent stem cells with the use of the TALEN expression vector upon Cel-I assay;

FIG. 9A depicts a partial base sequence of the clone ovomucoid gene locus and the amino acid sequence encoded by the base sequence for a knockout mutated clone;

FIG. 9B depicts a partial base sequence of the clone ovomucoid gene locus and the amino acid sequence encoded by the base sequence for a knockout mutated clone;

FIG. 9C depicts a partial base sequence of the clone ovomucoid gene locus and the amino acid sequence encoded by the base sequence for a knockout mutated clone;

FIG. 9D depicts a partial base sequence of the clone ovomucoid gene locus and the amino acid sequence encoded by the base sequence for the wild type;

FIG. 10 depicts colonies of the epiblast-derived pluripotent stem cell line having a cloned knockout mutation;

FIG. 11 depicts partial base sequences of the ovomucoid gene locus for epiblast-derived pluripotent stem cell lines each having a cloned knockout mutation and a wild type.

FIG. 12A depicts partial base sequence of the ovomucoid gene locus for epiblast-derived pluripotent stem cell line #4 having a cloned knockout mutation and partial sequence of the amino acid sequence encoded by the base sequence and the amino acid sequence encoded by the base sequence of the wild-type ovomucoid gene locus;

FIG. 12B depicts partial base sequences of the ovomucoid gene locus for epiblast-derived pluripotent stem cell lines #5 and #5-3 having a cloned knockout mutation and partial sequences of the amino acid sequence encoded by the base sequence and the amino acid sequence encoded by the base sequence of the wild-type ovomucoid gene locus;

FIG. 13A is a photo of a knockout chimeric chicken produced from an epiblast-derived pluripotent stem cell line #5 having a cloned knockout mutation;

FIG. 13B is a photo of a knockout chimeric chicken produced from an epiblast-derived pluripotent stem cell line #4 having a cloned knockout mutation;

FIG. 14 depicts the configuration of an ovomucoid knockout CRISPR/Cas9 vector;

FIG. 15 depicts the base sequences of oligo DNAs incorporated into a ovomucoid knockout CRISPR/Cas9 vector;

FIG. 16 depicts relative cleavage activity levels of ovomucoid knockout CRISPR/Cas9 vectors in HEK293 cells; and

FIG. 17 depicts relative cleavage activity levels of ovomucoid knockout CRISPR/Cas9 vectors in epiblast-derived pluripotent stem cells.

### DESCRIPTION OF EMBODIMENTS

Embodiments according to the present disclosure are explained. Note that the present disclosure is not limited to the embodiments described below and the drawings.

### Embodiment 1

At first, Embodiment 1 is explained. A bird according to Embodiment 1 lays an egg that does not contain an artificially introduced foreign gene and has an ovomucoid content lower than that in a wild type thereof.

Examples of a bird include, but are not particularly limited, chickens, ducks, turkeys, geese, wild geese, quails, pheasants, parrots, finches, hawks, ostriches, emus, and cassowaries. Preferably, the bird is a chicken. Examples of a chicken breed include, but are not particularly limited to, White Leghorn, Brown Leghorn, Barred Rock, Sussex, New Hampshire, Rhode Island, Ausstralorp, Minorca, Amrox, California Gray, Italian Partidge colored, and Korean Oge.

Genome of the bird according to this embodiment does not contain an artificially introduced foreign gene. The term “artificially introduced foreign gene” used herein means a gene having a mutation that is artificially introduced by gene recombinant technology or the like and a gene that is not originally contained in the genome of the bird. Examples of a method for artificially introducing a foreign gene into genome include a homologous recombination method, a retrovirus vector method, a lentivirus vector method, and an artificial viral vector method. The genome of the bird described above does not contain a foreign gene introduced by any of these methods.

Ovomucoid is a thermostable glycoprotein having a molecular weight of approximately 28000. Ovomucoid is produced by secretory cells in the oviduct. Usually, ovomucoid is mainly contained in albumen in bird eggs. Ovomucoid accounts for approximately 11% by weight of proteins contained in albumen in chicken eggs, for example. The ovomucoid content in an egg laid by the bird according to this embodiment is lower than that in a wild-type egg laid by a bird of the same kind. The term “wild type” used herein refers to a bird of the same kind of the above-described bird in which the gene is not artificially modified.

The ovomucoid content in an egg can be quantitatively determined by a known technique for detecting a target protein. For example, a sample prepared from albumen collected from a wild-type egg and a sample collected from an egg laid by the bird according to this embodiment are examined by immunostaining using an antibody that binds to ovomucoid according to the Western blot method. Thus, the ovomucoid content can be compared based on band intensity.

In order to ensure quantitative performance, the ovomucoid content is measured preferably by sandwich enzyme-linked immunosorbent assay (ELISA). A capture antibody and a detection antibody used in sandwich ELISA may be monoclonal antibodies or polyclonal antibodies. For example, a capture antibody and a detection antibody can be a rabbit anti-ovomucoid antibody and a mouse anti-ovomucoid antibody, respectively.

As a rabbit anti-ovomucoid antibody, a polyclonal antibody obtained by collecting anti-serum from a rabbit immunized with ovomucoid and purifying the anti-serum by affinity chromatography using ovomucoid. Meanwhile, as a mouse anti-ovomucoid antibody, a mouse anti-ovomucoid antibody, which is obtained by establishing a monoclonal antibody-producing hybridoma for ovomucoid by a cell fusion method using mouse spleen cells and purifying the resulting ascites antibody, may be used. A detection antibody is not particularly limited but may be labeled with peroxidase. In the case of labeling with peroxidase, ovomucoid can be quantitatively determined based on color development of TMB (3,3′,5,5′-tetramethylbenzidine). The ovomucoid content may be evaluated based on the concentration in a sample. Ovomucoid can be quantitatively determined at a detection limit of about 50 pg/ml by establishing adequate sandwich ELISA.

By quantitatively determining the ovomucoid content as described above, it is possible to confirm that the ovomucoid content in an egg laid by the bird according to this embodiment is reduced as compared with that for a wild-type bird of the same kind. For example, the ovomucoid content in an egg laid by a bird according to this embodiment is not more than 95% by weight, not more than 80% by weight, not more than 60% by weight, not more than 40% by weight, not more than 20% by weight, not more than 10% by weight, or not more than 5% by weight of the ovomucoid content in an egg laid by a wild-type bird of the same kind. In the case of quantitative determination of ovomucoid, the ovomucoid concentration in an egg laid by the bird according to this embodiment may be not more than the detection limit. In particular, in a case in which the bird according to this embodiment is a chicken, the ovomucoid content in an albumen protein in a chicken egg laid by the chicken may be 0% to 8% by weight, 0% to 4% by weight, 0% to 3% by weight, 0% to 2% by weight, or 0% to 1% by weight. The ovomucoid content may be evaluated based on the weight of ovomucoid per unit amount of albumen.

It is particularly preferable that an egg laid by the bird according to this embodiment does not contain ovomucoid. Note that the expression “not contain ovomucoid” means not containing ovomucoid that is contained in albumen in a wild-type egg. Therefore, eggs that do not contain ovomucoid also encompass eggs containing ovomucoid fragments other than a full-length ovomucoid fragment.

A knockout mutation that prevents expression of ovomucoid or a mutation that does not allow expression of full-length ovomucoid are present within the ovomucoid gene locus in the genome of the bird according to this embodiment. Such mutations do not allow normal expression of ovomucoid. The mutations may be optionally introduced unless ovomucoid is normally expressed. However, specifically, it is preferable to insert a termination codon into an exon within the ovomucoid gene locus.

The ovomucoid gene locus includes 5 exons. Assuming that the first to fifth exons from the 5′ end are exons 1 to 5, a termination codon may be inserted into any of exons 1 to 5. More preferably, a termination codon is included in at least one of exons 1 to 3 of the ovomucoid gene locus in the bird. When a termination codon is inserted into any of exons 1 to 5 within the ovomucoid gene locus, ovomucoid is not completely synthesized, thereby preventing full-length ovomucoid from being expressed. A partial fragment of ovomucoid is expressed depending on the site of insertion of a termination codon. However, allergenicity can be reduced as long as antigen epitopes of an ovomucoid fragment are reduced as compared with those of full-length ovomucoid.

In addition, the mutation may be a mutation of the initiation codon within the ovomucoid gene locus. In a case in which there is a mutation in an initiation codon within the ovomucoid gene locus, ovomucoid synthesis based on mRNA does not take place. For example, an initiation codon is not included in exon 1 of the ovomucoid gene locus in the bird.

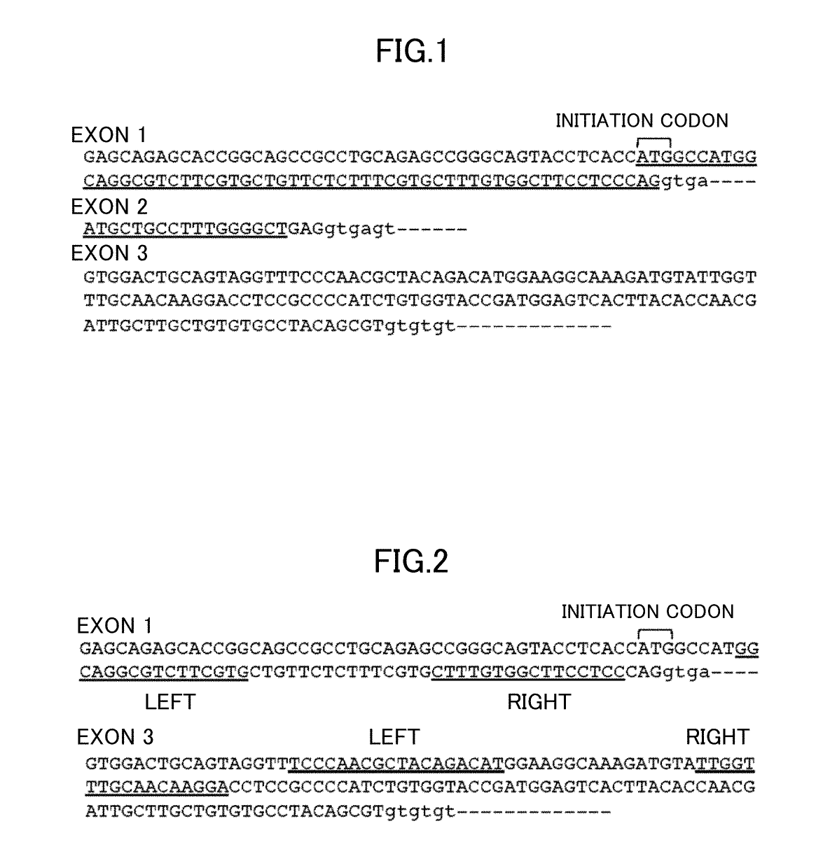

Preferably, the signal sequence of the ovomucoid gene locus is modified in the above-described bird. The signal sequence of the ovomucoid gene locus contains a partial base sequence of exon 1 and a partial base sequence of exon 2 and encodes an amino acid having 25 residues. FIG. 1 depicts the base sequences of exons 1, 2, and 3. Each upper-case letter denotes an exon base, and each lower-case letter denotes an intron base in FIG. 1. Each underlined base sequence in FIG. 1 is a signal sequence. Upon signal sequence modification, it is possible to introduce a mutation into ATG that is an initiation codon or introduce a mutation that causes generation of a termination codon in a signal sequence such that not only full-length ovomucoid but also ovomucoid fragments are not expressed. Preferably, a termination codon is included in exon 1 of the ovomucoid gene locus. The base sequences of exons 1, 2, and 3 are set forth in SEQ ID NOS: 1, 2, and 3, respectively.

As stated above, the bird does not contain an artificially introduced foreign gene in the genome. Therefore, it is preferable to use genome editing technology described in detail below but a homologous recombination method in which a specific gene on the genome is substituted by a foreign gene for production of the above-described bird in which the ovomucoid gene locus is modified.

As described in detail above, since ovomucoid is not expressed in a normal way in the bird according to this embodiment, the bird lays an egg in which the ovomucoid content is reduced as compared with that in a wild-type thereof. Since ovomucoid is highly allergenic, ovomucoid is independently tested in an egg allergy test, in addition to yolk and albumen. In view of this, an egg in which the ovomucoid content is lower than that in a wild type thereof, allergenicity in eggs can be sufficiently reduced.

In addition, the above-described bird does not contain an artificially introduced foreign gene in the genome. Since a foreign gene is not contained, the emergence of unpredictable phenotype and toxicity can be prevented. Further, since a foreign gene is not contained, loss of certainty of reproductive inheritance in the above-described bird can be prevented to a possible extent.

Moreover, since ovomucoid is highly physicochemically stable, allergenicity of ovomucoid is maintained even in heat-treated processed foods, vaccines or the like contain bird albumen. Accordingly, the egg in which the ovomucoid content is lower than that in a wild type thereof according to this embodiment is also useful as a raw material for various products such as processed foods and vaccines.

In addition, it was determined that the above-described bird may include a termination codon in at least one exon of exons 1 to 3 within the ovomucoid gene locus. In a case in which exon 3 includes a termination codon, antigen epitopes of a fragment of ovomucoid to be secreted are reduced as compared with those of full-length ovomucoid. Therefore, allergenicity can be reduced. In particular, in a case in which exon 1 in the ovomucoid gene locus includes a termination codon, the bird according to this embodiment can lay an egg, in which neither full-length ovomucoid nor ovomucoid fragment is contained. Further, in a case in which an initiation codon is not contained in exon 1 in the ovomucoid gene locus, ovomucoid is not synthesized based on mRNA. Therefore, the above-described bird can lay an egg containing no ovomucoid.

It was determined that a signal sequence within the ovomucoid gene locus may be modified in the above-described bird. A signal peptide corresponding to a signal sequence is cleaved in the intracellular endoplasmic reticulum and thus is not secreted. Therefore, in a case in which the signal sequence contains a termination codon, secretion of a peptide from the ovomucoid gene locus into albumen can be prevented. As a result, ovomucoid-derived allergenicity can be further reduced.

Note that the bird according to this embodiment was determined to be preferably a chicken. Since chickens lay highly marketable chicken eggs, chicken eggs having sufficiently reduced allergenicity can be efficiently supplied. In terms of supply of edible eggs, quails and the like are preferable as well as chickens.

A mutation within the ovomucoid gene locus may be, for example, a mutation that induces frameshift into an exon within the ovomucoid gene locus.

In another embodiment, a bird egg, which does not contain an artificially introduced foreign gene in genome thereof, and which has an ovomucoid content lower than that in a wild type thereof, is provided. In particular, chicken eggs are widely used as a raw material for confectionery, beverages, processed foods, and the like, or for production of pharmaceutical products such as vaccines. With the use of the chicken egg according to the embodiment, allergenicity of ovomucoid can be reduced even in a case in which ovomucoid is mixed in various products. Further, with the use of a bird eggs that does not contain ovomucoid, the risk of mixing ovomucoid having the strongest allergenicity in various products can be reduced to a possible extent.

### Embodiment 2

Next, Embodiment 2 is explained. In Embodiment 2, a method for producing a bird preferable for the bird according to Embodiment 1 is explained.

In order to produce a bird that lays an egg, in which the ovomucoid content is reduced as compared with that in a wild type thereof, it is necessary to produce a bird in which the ovomucoid gene locus is modified. For production a genetically modified animal, it is required to modify the genome of a one-cell stage fertilized egg and select mutated individuals of interest, from among the obtained individuals. Therefore, upon production of a genetically modified animal, it is necessary to manipulate a fertilized egg in vitro, thereby developing an individual from the fertilized egg. For example, it is possible to obtain a plurality of unfertilized eggs from one female mouse or rat. Therefore, after in-vitro fertilization, the development is proceeded to the one-cell stage to modify the genome and the fertilized egg is returned to the ovary of a female, thereby making it possible to produce a plurality of individuals.

Meanwhile, there is no established in-vitro fertilization technique for birds. In addition, only one one-cell stage fertilized egg can be obtained from only one bird that lays an egg. Further, it is difficult to identify a one-cell stage fertilized egg present in the oviduct. For the above reasons, it is difficult to apply genetic modification technology to a fertilized egg of a bird. Therefore, in order to produce a genetically modified bird, pluripotent stem cells having pluripotency or germinal differentiation capacity are used instead of fertilized eggs.

Therefore, the method for producing a bird according to this embodiment includes a modification step of cleaving the ovomucoid gene locus in bird pluripotent stem cells with programmable endonuclease so as to modify the ovomucoid gene locus and a transplantation step of transplanting the pluripotent stem cells in which the ovomucoid gene locus is modified into a bird embryo.

First, the modification step is described in detail. Examples of bird pluripotent stem cells include embryonic stem cells (ES cells) having pluripotency and germinal differentiation capacity. Bird ES cells are, for example, epiblast-derived stem cells (hereinafter also simply referred to as “epiSC”) that can be established from blastodermal cells isolated from the epiblast of a fertilized egg. In the case of chickens, the blastoderm in stage X of the development stages (I to XIV) of Eyal-Giladi and Kochav comprises epiblast. Chicken epiSC can be obtained by culturing blastodermal cells isolated from the epiblast on feeder cells, which were treated by irradiation or mitomycin C treatment so that the cell growth was suspended, by a known method. Examples of feeder cells include chicken embryo fibroblasts, mouse embryo fibroblasts, and a mouse embryo fibroblast-derived cell line.

Note that primordial germ cells may be used because they have pluripotency and germinal differentiation capacity. Primordial germ cells can be isolated from, for example, the fetal gonad of a bird by a known method. The case of using epiSC in the modification step is described below.

Upon modification of the ovomucoid gene locus, for example, the ovomucoid gene locus on the genome of epiSC is cleaved with programmable endonuclease at a specific site. Programmable endonuclease is used for so-called genome editing technology, by which a target site on the genome can be modified (deleted, substituted, or inserted) in a specific manner. Programmable endonuclease is designed based on the base sequence of target DNA such that DNA can be cleaved with an arbitrary base sequence. Examples of programmable endonuclease include, but are not particularly limited to, TALEN, zinc finger nuclease (ZFN), and Cas9 nuclease in the Clustered Regularly Interspaced Short Palindromic Repeat and Crisper associated protein (CRISPR-Cas) system (also referred to as “CRIPPR/Cas9”).

TALEN and ZFN are polypeptides each comprising a DNA-binding domain and a DNA cleavage site. A pair of DNA cleavage domains come close to form a dimer at a binding site of DNA-binding domains. Accordingly, TALEN and ZFN cleave double-strand DNA. The DNA-binding domain contain a repeat of a plurality of DNA-binding modules. Each DNA-binding module recognizes a specific base pair of DNA. Therefore, by designing an appropriate DNA-binding module, the base sequence that is a target of the ovomucoid gene locus can be cleaved in a specific manner.

In the CRISPR-Cas system, guide RNA, which has a base sequence complementary to the base sequence of a target that is adjacent to the PAM sequence on the genome, and Cas9 nuclease are used. Guide RNA includes CRISPR RNA (crRNA) complementary to a target base sequence and auxiliary tracrRNA. Cas9 nuclease, which has recognized guide RNA bound to a target base sequence of the ovomucoid gene locus, cleaves double-strand DNA in a region comprising a target base sequence on the 5′ end side of the PAM sequence.

Cleavage of double-strand DNA by programmable endonuclease may cause loss of lots of genetic information or canceration, and therefore, the cleaved site is very quickly repaired in cells. Upon non-homologous end joining repair, which is a major repair process for joining the cleaved ends, a mutation (deletion or insertion) is added to the genome base sequence with a high probability. Therefore, in the case of using TALEN, it is only required to design TALEN in accordance with the base sequences (effector sequences) of regions which are present on the 5′ and 3′ ends of a site to be modified within the ovomucoid gene locus and recognized by a DNA-binding module. Effector sequences preferable for a site to be modified within the ovomucoid gene locus can be identified by, for example, “TALEN Targeter” (https://tale-nt.cac.cornell.edu/). In addition, in the case of using the CRISPR-Cas system, a target base sequence of the ovomucoid gene can be identified by, for example, “CRISPR direct” (http://crispr.dbcls.jp/).

In the modification step, for example, a vector that expresses programmable endonuclease can be introduced into epiSC by a known method such as a microinjection method, an electroporation method, a calcium phosphate method, or a lipofection method. For example, in a case in which a TALEN expression vector is introduced as programmable endonuclease into epiSC, a TALEN expression vector designed based on the target base sequence of each double-strand of genome DNA is used. In a case in which the CRISPR-Cas system is used as programmable endonuclease, a vector that expresses guide RNA and a vector that expresses Cas9 nuclease can be inserted into epiSC in the same manner.

Upon modification of the ovomucoid gene locus, it is only required that a mutation is introduced into an arbitrary site within the ovomucoid gene locus and preferably at least one exon of exons 1 to 3 or a signal sequence such that the expression of ovomucoid is prevented. Preferably, the mutation causes at least one exon of exons 1 to 3 to include a termination codon or the mutation causes exon 1 not to include an initiation codon. Therefore, it is only required to design programmable endonuclease such that at least one exon of exons 1 to 3 or a signal sequence is cleaved. Programmable endonuclease is designed by a known method in accordance with the base sequence in the vicinity of the cleavage site.

Specifically, TALEN is TALEN left (first nuclease) or TALEN right (second nuclease). In the case of cleavage of exon 1, TALEN left and TALEN right comprise, for example, the amino acid sequence set forth in SEQ ID NO: 4 and the amino acid sequence set forth in SEQ ID NO: 5, respectively. In addition, in the case of cleavage of exon 3, TALEN left (third nuclease) and TALEN right (fourth nuclease) comprise, for example, the amino acid sequence set forth in SEQ ID NO: 6 and the amino acid sequence set forth in SEQ ID NO: 7, respectively.

TALEN left may comprise an amino acid sequence derived from the amino acid sequence set forth in SEQ ID NO: 4 or SEQ ID NO: 6 by deletion, substitution, or addition of one or several amino acids as long as it has nuclease activity that allows specific cleavage of a target base sequence. TALEN right may also comprise an amino acid sequence derived from the amino acid sequence set forth in SEQ ID NO: 5 or SEQ ID NO: 7 by deletion, substitution, or addition of one or several amino acids as long as it has nuclease activity that allows specific cleavage of a target base sequence.

Meanwhile, in the CRISPR-Cas system for cleavage of exon 1, for example, Cas9 nuclease may cleave double-strand DNA in a region comprising a base sequence set forth in any one of SEQ ID NOS: 8 to 11 within the ovomucoid gene locus. In addition, in the case of cleavage of exon 2, Cas9 nuclease may cleave double-strand DNA in a region comprising a base sequence set forth in SEQ ID NO: 12 or 13 within the ovomucoid gene locus.

Oligo DNA that causes guide RNA to be expressed is preferably used for introducing guide RNA into epiSC. The base sequence of oligo DNA is determined based on a target base sequence. In a case in which Cas9 nuclease cleaves a region comprising the base sequence set forth in SEQ ID NO: 8, the sense base sequence of oligo DNA that causes guide RNA to be expressed in epiSC includes the base sequence set forth in SEQ ID NO: 14, and the antisense base sequence of the oligo DNA includes the base sequence set forth in SEQ ID NO: 15. In addition to the above, in a case in which the base sequence of a region that is cleaved by Cas9 nuclease is designated as the base sequence set forth in any one of SEQ ID NOS: 9, 10, and 11, examples of a combination of a base sequence included in the sense base sequence and a base sequence included in the antisense base sequence of oligo DNA are a combination of SEQ ID NO: 16 and SEQ ID NO: 17, a combination of SEQ ID NO: 18 and SEQ ID NO: 19, and a combination of SEQ ID NO: 20 and SEQ ID NO: 21. Further, in a case in which the base sequence of a region that is cleaved by Cas9 nuclease is designated as the base sequence set forth in SEQ ID NO: 12 or 13, examples of a combination of a base sequence included in the sense base sequence and a base sequence included in the antisense base sequence of oligo DNA are a combination of SEQ ID NO: 22 and SEQ ID NO: 23, and a combination of SEQ ID NO: 24 and SEQ ID NO: 25.

It is possible to judge whether or not the ovomucoid gene locus has been modified in the above-described modification step by analyzing the base sequence of the ovomucoid gene locus on the genome of epiSC. For example, after the introduction of programmable endonuclease, genome DNA can be recovered from stably growing epiSC, thereby analyzing the base sequence of the ovomucoid gene locus.

In order to efficiently obtain a chimeric individual having the genome that has been modified such that ovomucoid is not expressed, epiSC, in which a knockout mutation that causes the ovomucoid gene not to be expressed has been introduced into the ovomucoid gene locus, may be selected. Alternatively, in order to concentrate cells, into which a vector that expresses programmable endonuclease has been introduced, an expression system in which a drug-resistant gene such as puromycin is transiently expressed, may be introduced into epiSC.

Next, the transplantation step is described in detail. In the transplantation step, epiSC, in which the ovomucoid gene locus has been modified, is transplanted into a bird embryo. The transplantation operation is not particularly limited. However, epiSC can be injected into a bird embryo using a narrow tube.

Specifically, in the transplantation step, for example, epiSC, in which the ovomucoid gene locus has been modified to cause ovomucoid not to be expressed, can be transplanted to the blastoderm of a gamma-irradiated fertilized egg embryo immediately after oviposition. In this case, it is possible to readily discriminate a chimeric individual based on feather color using a recipient of a cell line with a feather color different from that of the cell line of epiSC. For example, in the case of production of a chimeric chicken, it is preferable that epiSC from the Barred Plymouth Rock variety with black feathering in the chick phase is transplanted into an embryo of the White Leghorn variety with white feathering in the chick phase.

Following the transplantation step, eggs including the embryo, into which epiSC has been transplanted, are hatched, thereby making it possible to produce chimeric individuals. The period of incubation is approximately 20 days for chickens. In chimeric individuals, a sperm and an ovum having the genome including the modified ovomucoid gene locus are formed. Therefore, by crossing chimeric individuals, a genetically modified bird, which has inherited the genome including the modified ovomucoid gene locus in the homozygous form, can be produced with a high probability. Since the ovomucoid gene locus of the genetically modified bird has been modified, the ovomucoid content in an egg laid by the genetically modified bird is lower than that in a wild type thereof or an egg laid by the genetically modified bird does not contain ovomucoid.

Note that such genetically modified bird can be distinguished based on the feather color as described above. In addition, such genetically modified bird can be discriminated by analyzing the base sequence of genome DNA thereof by Southern blot or the like.

As described in detail above, according to the method for producing a bird according to this embodiment, the ovomucoid gene locus in a bird pluripotent stem cell is cleaved with programmable endonuclease so as to be modified. Thus, a bird in which ovomucoid cannot be normally expressed can be obtained. Since the genome of the bird is inheritable through the reproductive process, the ovomucoid content in an egg laid by the bird is reduced as compared with that in a wild type thereof. Alternatively, an egg laid by the bird does not contain ovomucoid. Accordingly, allergenicity of the egg can be sufficiently reduced.

In addition, in the modification step in this embodiment, the ovomucoid gene locus of epiSC may be modified. It is readily possible to establish epiSC from blastodermal cells which can be obtained in an amount of approximately 60,000 cells from a single embryo and to stably culture epiSC while maintaining pluripotency, thereby making it possible to modify ovomucoid gene locus with improved certainty.

### EXAMPLES

The present disclosure is more specifically explained with reference to the Examples below. However, the present disclosure is not limited to the Examples.

### Example 1: Production of TALEN Expression Vector

(Selection of Target Sequences of Ovomucoid)

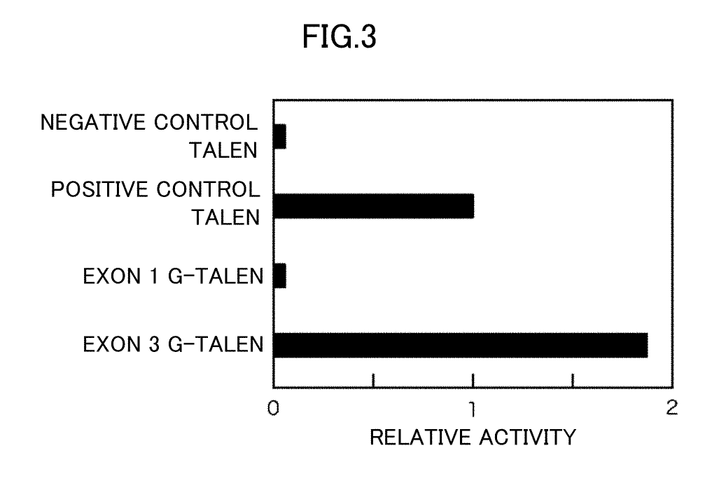

In order to mutate the ovomucoid gene by TALEN, an effector sequence was searched for by TALEN Targeter for the base sequences of exons 1, 2, and 3 of ovomucoid. As a result, as indicated by underlined regions in FIG. 2, a set of effector sequences was found in each of the base sequences of exons 1 and 3. Meanwhile, no effector sequence was found in exon 2.

(Production of TALEN Expression Vectors and Examination of Cleavage Activity of TALEN)

At first, modules capable of binding to the respective effector sequences of exons 1 and 3 were constructed by the 6-module assembly method, thereby preparing two types of Golden Gate TALEN expression vectors (left and right) for each of exons 1 and 3. Golden Gate TALEN expression vectors (hereinafter also simply referred to as “G-TALEN expression vectors”) were prepared using a Golden Gate TALEN and TAL Effector Kit 2.0 and a Yamamoto Lab TALEN Accessory Pack (both are available from Addgene) in accordance with the protocols attached to the kits.

Next, in order to examine cleavage activity of TALEN, single-strand annealing (SSA) assay was conducted using HEK293 cells. Upon SSA assay, HEK293 cells were cotransfected with a reporter vector having a base sequence serving as a target of TALEN and a G-TALEN expression vector, and cleavage activity was determined based on reporter activity as described below.

The reporter vector was prepared by inserting synthetic oligo annealed to pGL4-SSA contained in the Yamamoto Lab TALEN Accessory Pack. At first, pGL4-SSA was treated with BsaI, electrophoresed without dephosphorylation, and excised. Regarding exon 1, the base sequences of sense oligo and antisense oligo included in the inserted synthetic oligo are set forth in SEQ ID NO: 26 and SEQ ID NO: 27, respectively. Regarding exon 3, the base sequences of sense oligo and antisense oligo included in the inserted synthetic oligo are set forth in SEQ ID NO: 28 and SEQ ID NO: 29, respectively.

Here, details of the synthetic oligo annealing solution are described below. 10×Buffer: 1 μl (400 mM Tris-HCL (pH8), 200 mM MgCl₂, 500 mM NaCl)

Sense oligo (50 μM): 1 μM
Antisense oligo (50 μM) 1 μM
Sterile distilled water: 7 μM

The annealing solution was maintained at 95° C. for 5 minutes, and then cooled to 25° C. over 90 minutes, thereby annealing synthetic oligo.

Next, the annealed synthetic oligo was inserted into BsaI-treated pGL4-SSA. A small culture of the subcloned product was obtained and treated with KpnI. Accordingly, two bands, which were a 3800-bp band and a 1800-bp band, appeared, confirming that the synthetic oligo was inserted.

Next, sequence analysis of the reporter vector was conducted by the following procedures. The reporter vector was treated with NarI and electrophoresed. Thereafter, gel was excised and a gel fragment was collected into a microtube. The microtube was placed in a deep freezer for about 10 minutes such that the gel was frozen. Then, the gal was thawed by hand warming and then spun down by a centrifuge. The resulting leachate in an amount of 6 to 8 μl was collected and used as a sequence template.

Primers used for sequence analysis were Luc2-up-F (SEQ ID NO: 30) and Luc2-down-R (SEQ ID NO: 31). After the confirmation of insertion of a correct base sequence, the reporter vector was purified using a transfection-grade Miniprep kit. The concentration was quantitatively determined and adjusted to 150 ng/μl.

HEK293 cells were transfected with a DNA solution prepared by mixing the following 4 types of plasmids.

| | |
|---|---|
| G-TALEN expression vector (Left) | 200 ng |
| G-TALEN expression vector (Right) | 200 ng |
| Reporter vector | 100 ng |
| pRL-CMV (reference vector) | 20 ng |

HEK293 cells were cultured at 70% to 80% confluency in a 10-cm culture dish. Serum-free Dulbecco's Modified Eagle's Medium (hereinafter referred to as “DMEM”) for DNA dilution and serum-free DMEM for Lipofectamine LTX dilution were separately dispensed in required amounts into microtubes. Serum-free DMEM for DNA dilution was added in an amount of 25 μl to each well of a 96-well plate, and 4 to 8 μl of the above-described DNA solution was added to each well, followed by mixing. LTX was added to serum-free DMEM for LTX dilution in an amount of 0.7 μl per well (250) and suspended therein, and the suspension was immediately added to each well in an amount of 25 μl, followed by mixing. The above process was repeated until the necessary number of tubes were prepared. The medium was removed from cells in the culture dish, 15% fetal bovine serum (hereinafter referred to as “FBS”)/DMEM was added thereto, and pipetting was performed directly over the culture dish, thereby suspending cells. The number of cells was counted using a cell counter plate and adjusted to 6×10⁵ cells/ml.

Prepared cells were added in an amount of 100 μl per well to each well 30 minutes after the addition of LTX to the first wells, and the cells were incubated in a CO₂ incubator at 37° C. Luciferase activity was measured 24 hours after transfection using the Dual-Glo Luciferase Assay System (manufactured by Promega Corporation) in accordance with the manufacturer's instructions.

(Results)

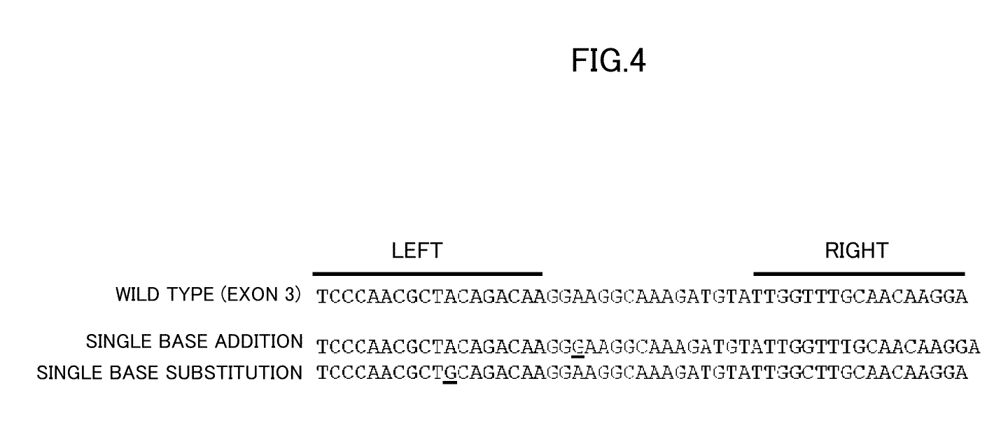

FIG. 3 depicts cleavage activity levels of G-TALEN expression vectors obtained by SSA assay. Positive control TALEN is a TALEN expression vector having sufficient cleavage activity prepared for targeting HPRT1. Relative activity is a value relative to cleavage activity of HPRT1 in HEK293 cells, provided that the measured cleavage activity is 1. In the case of a negative control, a relative activity value obtained by introducing a G-TALEN expression vector into HEK293 cells free from a target sequence is obtained. As depicted in FIG. 3, the G-TALEN expression vector targeting exon 1 did not have cleavage activity while cleavage activity was exclusively confirmed in the G-TALEN expression vector targeting exon 3.

### Example 2: Mutagenesis in Chicken epiSC with the G-TALEN Expression Vector and Confirmation of Mutagenesis by Cel-I Assay

The G-TALEN expression vector targeting exon 3, which was confirmed to have cleavage activity, was used for mutagenesis of chicken epiSC.

(Culture of Chicken epiSC)

First, blastodermal cells were separated from a fresh fertilized egg obtained immediately after oviposition by the following procedures. Albumen was completely removed by an egg separator and a fertilized egg was allowed to stand still in a plastic petri dish such that the blastoderm was positioned on the yolk. A sterilized and dried filter paper ring (prepared by making a 5-mm hole on filter paper and cutting the filter paper along the outer ring of the hole) was applied to the fertilized egg such that the blastoderm was positioned at the center of the ring. The filter paper ring was cut by scissors (small straight scissors with sharp points) along the outer circumference of the ring and the blastoderm was roundly cut together with yolk membrane. Subsequently, the filter paper was slowly raised obliquely with forceps, and yolk adhering the filter paper ring was removed to a possible extent. At such time, the epiblast is adhering to the filter paper ring.

Then, the filter paper ring was immersed in a petri dish containing sterile phosphate buffered saline (PBS) while the yolk side was upward. The filter paper ring was slowly shaken, thereby removing the adhering yolk. The filter paper ring was transferred to a separately prepared petri dish containing sterile PBS, and vigorously shaken for a while, thereby causing blastodermal cells to be separated in a disc form from the filter paper ring. The thus separated blastodermal cells were collected in a 1.5-ml tube by a micropipette.

Next, the separated blastodermal cells were cultured on preliminarily prepared feeder cells. A mouse embryo fibroblast-derived cell line (STO cells) was used as feeder cells. Preparation of feeder cells is explained below.

STO cells were seeded on a 10-cm culture dish. The culture solution was 10% fetal bovine serum (FBS)—Dulbecco's Modified Eagle medium (DMEM). Culture was conducted at 5% CO₂ and 37° C. STO cells reached confluent after culture for approximately 3 days and washed with cold PBS three times. The cells were detached using 0.025% trypsin and 0.02% EDTA 2Na-PBS, and seeded in a 15-cm culture dish. After the cells reached confluent, mitomycin C was added to the culture solution to result in a final concentration of 10 μg/ml, and the cells were cultured for 2 hours. The cells were washed with cold PBS five times, detached using 0.025% trypsin and 0.02% EDTA 2Na-PBS, and washed by centrifugation at least three times. The cell count was calculated using a cell counter plate.

Gelatin coating in a 6-cm culture dish was used for culture of feeder cells. A gelatin coating solution was prepared before use by adding gelatin to distilled water so as to result in a concentration of 0.1%, and autoclaving the resulting solution for dissolution and sterilization. A culture dish was coated at least 2 hours before culture of feeder cells such that the bottom face thereof was immersed in the gelatin coating solution at 37° C. After removal of the gelatin coating solution, the above-described mitomycin C-treated feeder cells were adjusted with 10% FBS-DMEM and seeded such that 2 to 3×10⁵ cells per 6-cm culture dish was achieved. The feeder cells were used within 5 days from the day following seeding.

(Mutagenesis in Chicken epiSC)

Blastodermal cells separated from one embryo were cultured in a culture dish in which feeder cells had been seeded. Table 1 lists the composition of medium used for culture of blastodermal cells. Note that the medium was prepared based on KnockOut-DMEM in Table 1, and the recombinant chicken leukemia inhibitory factor (recombinant chicken LIF) was added to a warmed medium in a minimum required amount immediately before use.

Here, a method for preparing recombinant chicken LIF is explained. The CHCC-OU2 chicken embryonic cell line was cultured with low-glucose DMEM (manufactured by Invitrogen) containing 10% FBS (Hyclone; manufactured by Thermo Fisher Scientific Inc.), 100 μg/ml penicillin, and 70 μg/ml streptomycin in 5% CO₂ at 37° C. The coding region of LIF was amplified by PCR using a forward primer set forth in SEQ ID NO: 32 and a reverse primer set forth in SEQ ID NO: 33. The PCR product was treated with NheI and SalI and subcloned into a pSecTag2A plasmid (manufactured by Invitrogen) including a histidine tag.

Next, myc-epitope was removed from the pSecTag2A plasmid using a restriction enzyme. The recombinant plasmid was introduced into CHCC-OU2 using Polyfect Transfection Reagent (manufactured by Qiagen), and cells were selected using a medium containing 0.25 μg/ml Zeocin (manufactured by Invitrogen). A stable cell line secreting LIF having biological activity was selected, and recombinant chicken LIF was purified from the culture supernatant using ProBond resin (manufactured by Invitrogen).

TABLE 1

| | Final |
|---|---|
| Reagent name (Manufacturer, Catalog No.) | concentration |
| KnockOut serum replacement (Invitrogen #10828-028) | 20% |
| chicken serum (Invitrogen #16110-082) | 2% |
| sodium pyruvate (Gibco #11360-070) | 1% |
| MEM NEAA (Gibco #11140-050) | 1% |
| GlutaMax (Gibco #35050-061) | 1% |
| 100X nucleosides (Millipore #ES-008D) | 1% |
| Antibiotic-Antimycotic mixed | 1% |
| stock solution (nacalai tesque #09366-44) | |
| β-mercaptoethanol (Sigma #M7522) | 0.1 mM |
| KnockOut-DMEM (Invitrogen #10892-018) | 20 ng/mL |
| recombinant chicken LIE | |

A G-TALEN expression vector in an amount of 6.5 μg was introducing into epiSC obtained by culturing the blastodermal cells on the feeder cells for 2 to 3 days using FuGENE HD (manufactured by Promega). Puromycin was added to the medium so as to result in a concentration of 2 μg/mL 24 hours after the introduction of the G-TALEN expression vector, followed by culture for 48 hours. After the elapse of 48 hours, the medium was refreshed, thereby removing puromycin from the medium. Culture was carried out for approximately 10 days until stable growth of epiSC was confirmed.

Genome DNA was recovered from stably grown epiSC using a DNeasy Blood & Tissue Kit (manufactured by QIAGEN), followed by genomic PCR. Genomic PCR included 94° C. for 2 minutes and 35 cycles of 98° C. for 10 seconds, 68° C. for 30 seconds, and 72° C. for 2 minutes. The base sequences of a forward primer and a reverse primer used in genomic PCR are set forth in SEQ ID NO: 34 and SEQ ID NO: 35, respectively.

(Cel-I Assay)

Subsequently, Cel-I assay involving rehybridization of the PCR product, treatment with surveyor nuclease, and cleavage at the heteroduplex site was conducted. A SURVEYOR (trademark) Mutation Detection Kit (manufactured by Transgenomic, Inc.) was used for Cel-I assay. The PCR product was purified using a Wizard SV Gel and PCR Clean-up System (manufactured by Promega). DNA was eluted in an amount of 15 μl. After elution, the DNA concentration was quantitatively determined.

Next, a DNA solution for Cel-I assay was prepared based on the following composition.

PCR product 400 ng

10×Hybridization buffer (100 mM Tris-HCl (pH 8.5), 750 mM KCl, 15 mM MgCl₂) 0.8 μl

Adjustment with sterile distilled water to 8 μl

The DNA solution was maintained at 95° C. for 5 minutes and cooled to 25° C. over 60 to 90 minutes.

To the DNA solution, 0.4 μl of Enhancer S and 0.4 μl of Nuclease S were added, pipetting was conducted to a sufficient extent, and the mixture was incubated at 42° C. for 30 minutes. Immediately after the reaction, the full amount of the mixture was electrophoresed using agarose gel or polyacrylamide gel.

(Results)

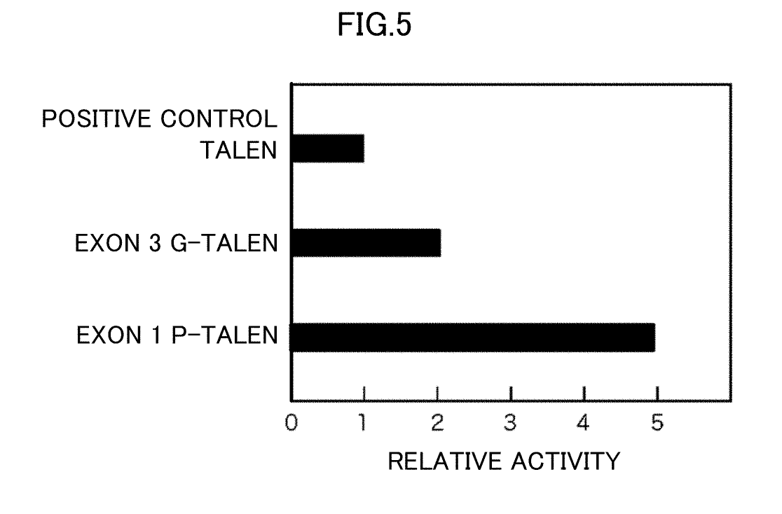

No clear band indicating mutagenesis was obtained in Cel-I assay. Meanwhile, as a result of analysis of the base sequence of the PCR product, two kinds of mutation, which were single base addition and single base substitution, were observed in the target region as depicted in FIG. 4 (see the underlined portions). However, none of these mutations was a mutation that causes generation of a termination codon.

### Example 3: Preparation of Platinum Gate TALEN Expression Vector and Evaluation of Cleavage Activity

A Platinum Gate TALEN (hereinafter also simply referred to as “P-TALEN”) expression vector targeting the above-described base sequence of exon 1, which is high activity-type TALEN, was prepared. A module capable of binding to the effector sequence in exon 1 was constructed by the 6-module assembly method, thereby preparing P-TALEN expression vectors (left and right) for exon 1. The P-TALEN expression vector was prepared using a Platinum Gate TALEN Kit and a Yamamoto Lab TALEN Accessory Pack (both are available from Addgene) in accordance with the protocols attached to the kits. Cleavage activity of the P-TALEN expression vector was evaluated by SSA assay in the manner described above.

(Results)

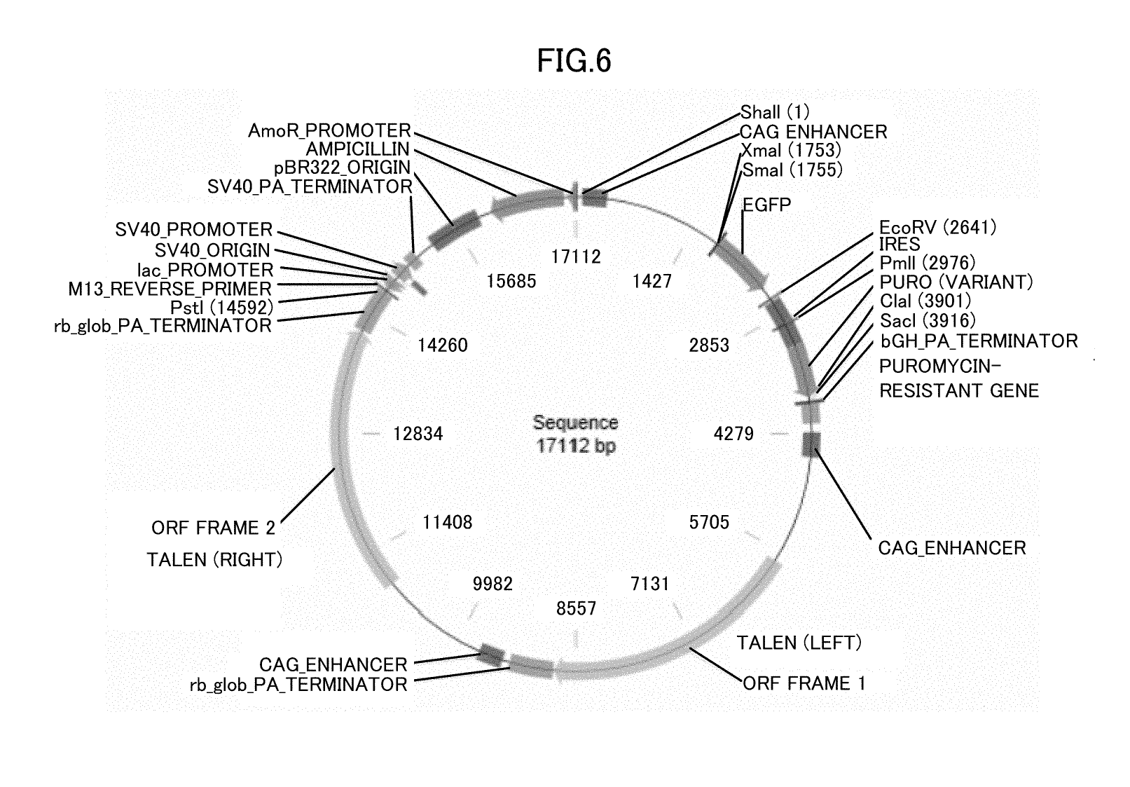

FIG. 5 depicts cleavage activity of the P-TALEN expression vector in SSA assay. The P-TALEN expression vector was confirmed to have cleavage activity, which was greater than that of the G-TALEN expression vector targeting exon 3 prepared in Example 1.

### Example 4: Preparation of all-in-One P-TALEN Vector and Mutagenesis in Chicken epiSC

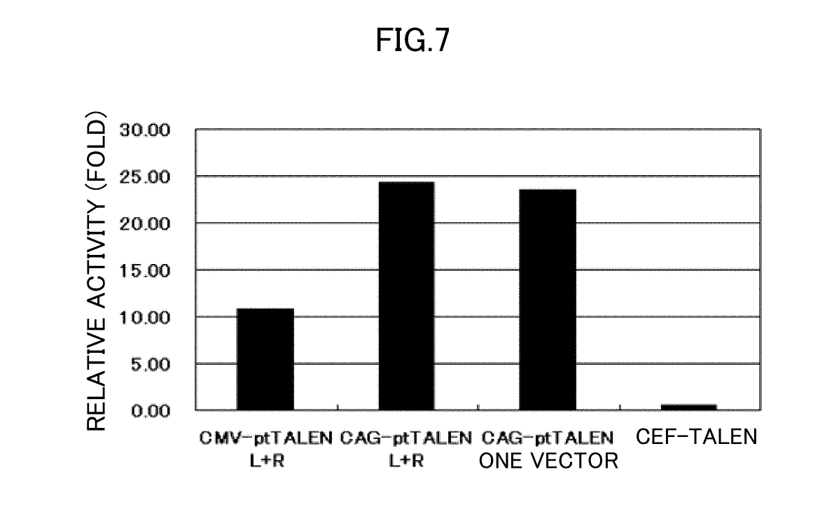

In order to improve P-TALEN expression vector transfection efficiency, two kinds of vectors, which were a P-TALEN expression vector (Left) and a P-TALEN expression vector (Right), were integrated into a one vector. In addition, in order to transiently concentrate cells transfected with the vector, a puromycin-resistant gene expression cassette was introduced into the one vector. FIG. 6 depicts the configuration of the constructed one vector. Cleavage activity of the constructed one vector was evaluated by SSA assay in the manner described above. Chicken embryo fibroblasts (CEFs) were used for evaluating cleavage activity in chicken cells in SSA assay

Cultured epiSC was transfected with 6.5 μg of the one vector using FuGENE HD (manufactured by Promega). In order to improve mutagenesis efficiency independently from the transfection with the one vector alone, epiSC was transfected with a chicken exonuclease I expression vector (EXO I) together with the one vector. Puromycin was added to the medium so as to result in a concentration of 2 μg/mL 24 hours after the introduction of the one vector, followed by culture for 48 hours. After the elapse of 48 hours, the medium was refreshed, thereby removing puromycin from the medium. Culture was carried out for approximately 10 days until stable growth of epiSC was confirmed.

Genome DNA was recovered from epiSC and genomic PCR was conducted in the manner described above. The base sequences of a forward primer and a reverse primer used in genomic PCR are set forth in SEQ ID NO: 36 and SEQ ID NO: 37, respectively. Further, Cel-I assay was conducted using the PCR product.

(Results)

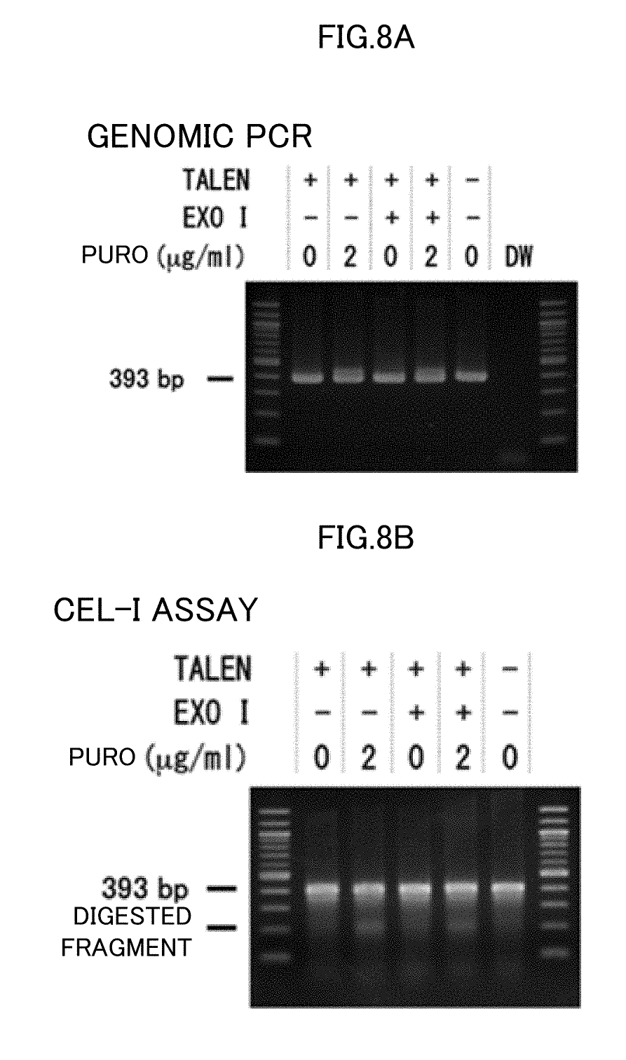

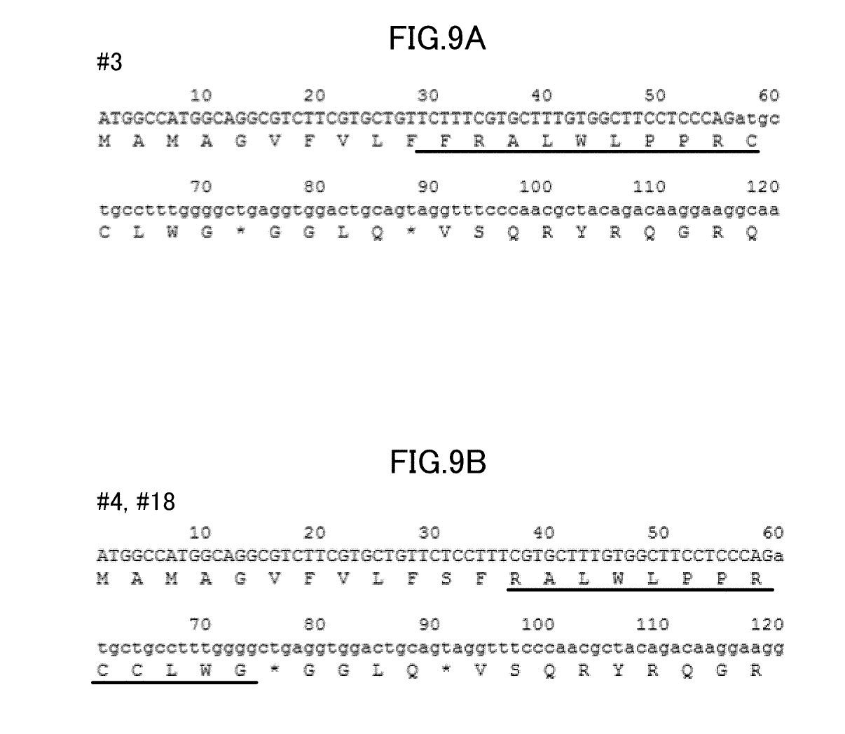

FIG. 7 depicts cleavage activity of the one vector in SSA assay. Relative activity is a value relative to a measurement value of 1 in a case in which CEFs free of the target sequence were transfected with the one vector. As depicted in FIG. 7, the one vector was found to have sufficient cleavage activity. Note that the expression “CMV-ptTALEN L+R” refers to a vector controlling the expression of Left and Right TALENs by a CMV promoter, and the expression “CAG-ptTALEN L+R” refers to a vector controlling the expression of Left and Right TALENs by a CAG promoter. Upon genomic PCR, a shift band indicating a heteroduplex generated through mutation in the drug-based selection system was observed as depicted in FIG. 8A. Upon Cel-I assay, a band of a digested fragment indicating mutagenesis was observed in the genome of epiSC obtained via drug selection as depicted in FIG. 8B. No effects of introduction of chicken EXO I were confirmed.

### Example 5: Introduction of Knockout Mutation into epiSC and Cloning

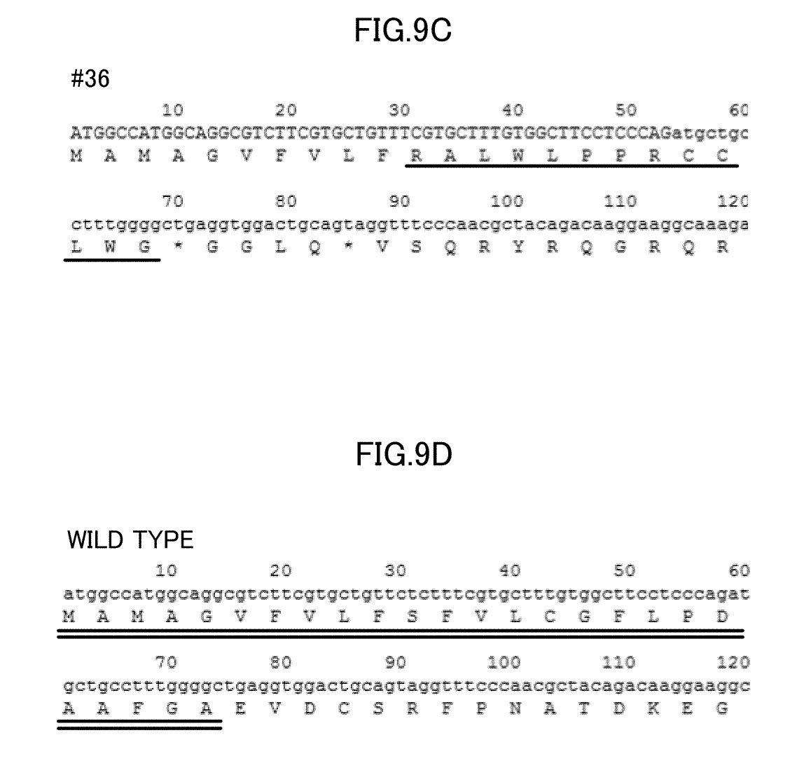

The mutated region in genome DNA of epiSC described above was amplified by PCR and cloned into a vector. Then, the base sequence of the mutated region was analyzed. As a result of analysis of 43 clones, deletion was found in 10 clones, insertion was found in 1 clone, and substitution was found in 2 clones. Mutagenesis efficiency was as high as 30%. As a result of analysis of knockout mutation, a knockout mutation due to insertion of the termination codon was detected in 3 clones (deletion for 2 clones and insertion for 1 clone). As representative mutated base sequences, #3 (FIG. 9A) and #36 (FIG. 9C), in which with deletion was observed, and #4 and #18 (FIG. 9B), in which insertion was observed, are exemplified. In FIG. 9D, the double underlined portion of the wild type indicates a signal sequence. The underlined portions of #3, #18 and #36 each indicate an amino acid sequence mutated through mutagenesis in FIGS. 9A, 9B and 9C, respectively. Efficiency of knockout mutation was 7%.

After transfection of epiSC with the one vector, 3300 cells were seeded on 96-well plates, followed by cloning. epiSC colonies were grown in 49 wells of 9 plates in total, and eventually the growth was successfully completed in 27 wells. Cells in 27 wells were cryopreserved, and genome was partially extracted from each cell. Cel-I assay was conducted in the manner described above. Further, base sequence analysis was conducted.

(Results)

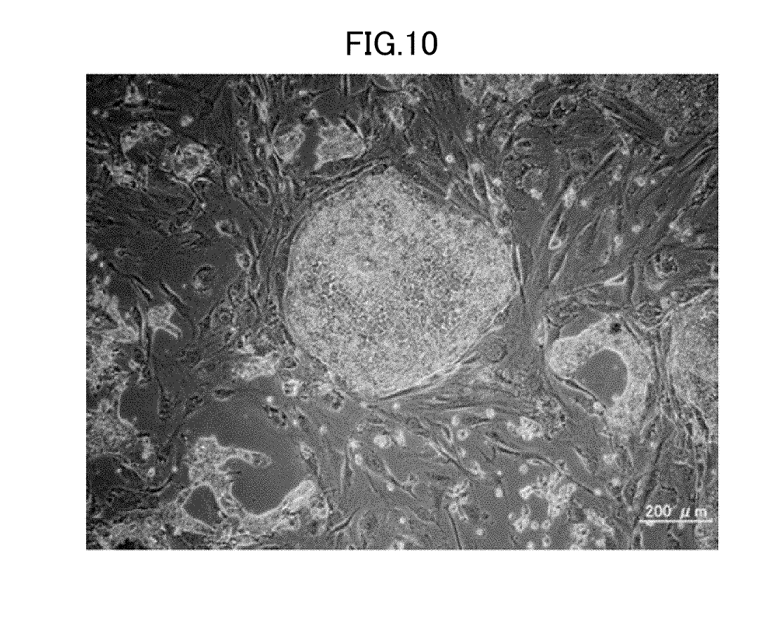

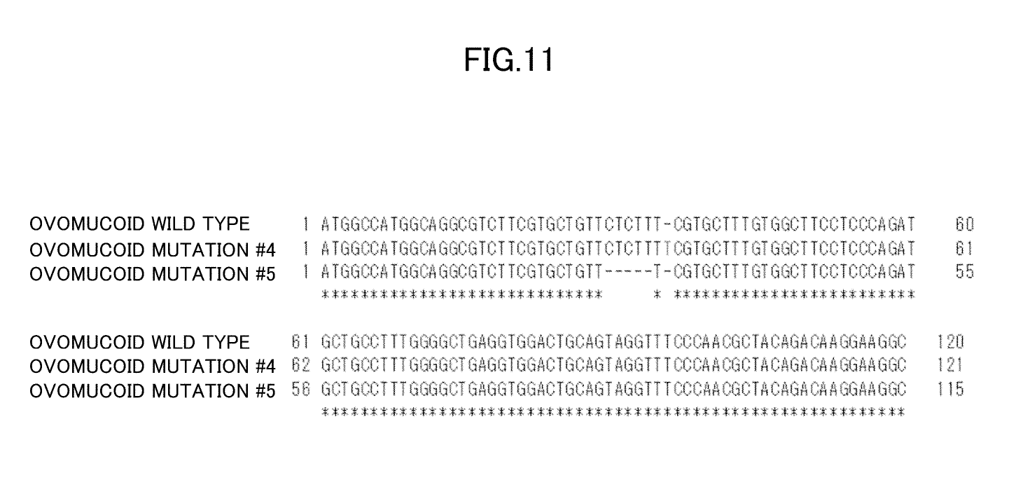

Positive results of Cel-I assay were confirmed in 4 out of 27 wells. After stable growth of the cells as in the case of clone #5 exemplified in FIG. 10, base sequence analysis was conducted. As depicted in FIG. 11, there was an insertion of “T” next to the 35th base from the 5′ end of the base sequence of the mutated region of the cloned ovomucoid knockout epiSC cell line #4. This base sequence was converted into an amino acid sequence. As a result, it was revealed that the termination codon is inserted into positions corresponding to the 26th and 31st residues from the N-terminus.

Meanwhile, in the base sequence of the mutated region of the ovomucoid knockout epiSC cell line #5, there was a deletion of 5 bases at the position corresponding to the base sequence depicted in FIG. 9C. This base sequence was converted into an amino acid sequence. As a result, it was revealed that the termination codon is inserted into positions corresponding to the 24th and 29th residues from the N-terminus.

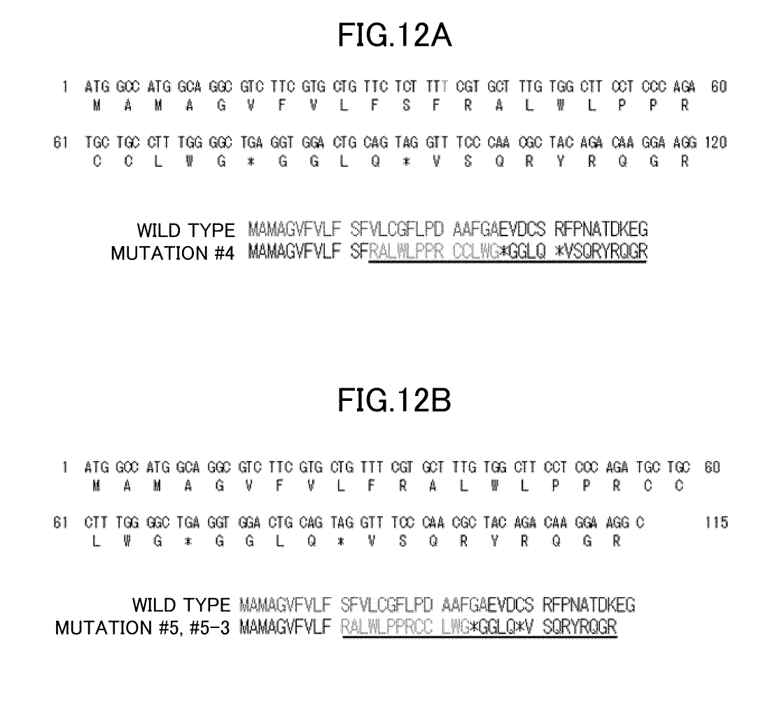

The amino acid sequence encoded by the base sequence of the epiSC cell line #4 was compared with the amino acid sequence of the wild type ovomucoid. As a result, it was found that frameshift causes an amino acid mutation from the 13th residue from the N-terminus of the signal peptide, and translation proceeds to the 25th residue of the signal peptide as depicted in FIG. 12A. A signal peptide is cleaved in the intracellular endoplasmic reticulum and thus not secreted. Therefore, this mutation was confirmed to be a knockout mutation of ovomucoid.

The amino acid sequence encoded by the base sequence of the epiSC cell line #5 was compared with the amino acid sequence of the wild type ovomucoid. As a result, it was found that frameshift causes an amino acid mutation from the 11th residue from the N-terminus of the signal peptide, and translation proceeds to the 23th residue of the signal peptide as depicted in FIG. 12B. The mutation was confirmed to be a knockout mutation of ovomucoid also for the epiSC cell line #5. Note that since no other base sequences were observed in the ovomucoid knockout epiSC cell line #5 as a result of base sequence analysis, complete cloning of the ovomucoid knockout epiSC cell line #5 was confirmed. However, for further confirmation, cloning was conducted once again, and the ovomucoid knockout epiSC cell line #5-3 was also prepared.

### Example 6: Production of Chimeric Chickens

The ovomucoid knockout epiSC cell lines #4, #5, and #5-3 were each transplanted into the blastoderm of a 5-Gy gamma-irradiated fertilized egg embryo immediately after oviposition, thereby hatching germline chimeric chickens (G0).

(Results)

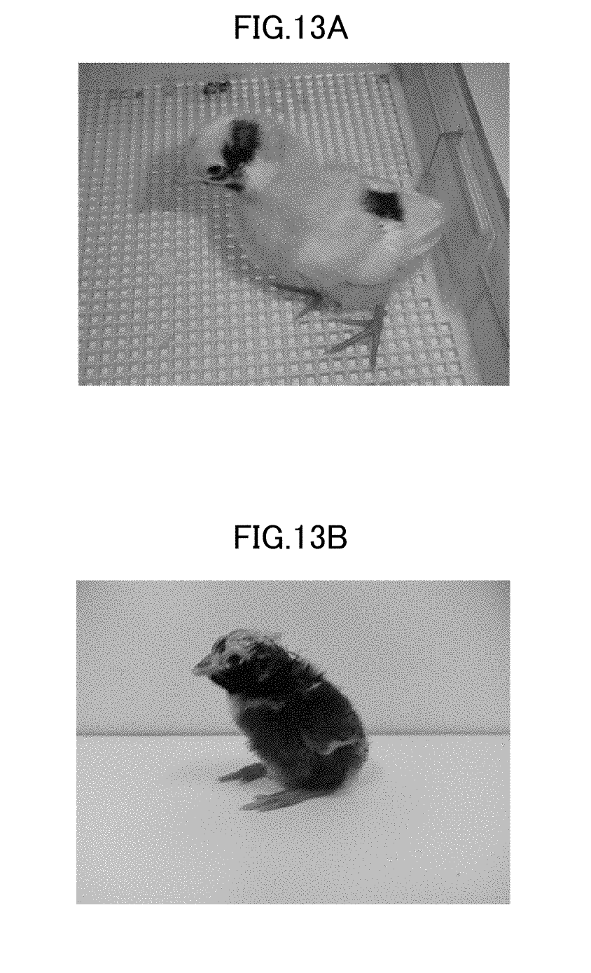

As a result, 18 chimeric chickens were produced. The ovomucoid knockout epiSC cell line is of the Barred Plymouth Rock variety (black feathering in the chick stage), and a transplantation recipient embryo is of the White Leghorn variety (white feathering in the chick stage). Therefore, in a chimera thereof, ovomucoid knockout epiSC is differentiated into epidermis, which results in black feathering. FIGS. 13A and 13B depict the appearance of an epiSC cell line #5-derived chimeric chicken and the appearance of an epiSC cell line #4-derived chimeric chicken, respectively. These chimeric chickens were observed with black feathering. As listed in Table 2, the breakdown of chimeric chickens consisted of 6 males, 7 females, and 5 unknown individuals. Among 18 chickens, 11 chickens were black feather chimeras. The proportion of black feathering is shown in each “Feathering” column in Table 2.

TABLE 2

| | | Date of | | | |
|---|---|---|---|---|---|
| Serial No. | ID No. | hatching | epiSC | Male/Female | Feathering |
| 1 | 1 | 20140930 | #5 | ♂ | Black (15%) |
| 2 | 2 | 20141017 | #5 | ♂ | Black (5%) |
| 3 | 7289 | 20141111 | #5 | ♂ | — |
| 4 | 7292 | 20141230 | #5-3 | ♀ | Black (5%) |
| 5 | 7293 | 20150106 | #5 | ♀ | Black (1%) |
| 6 | 7294 | 20150407 | #5 | ♂ | Black (30%) |
| 7 | 7295 | 20150407 | #5 | ♀ | — |
| 8 | 7296 | 20150407 | #5 | ♂ | — |
| 9 | 7606 | 20150414 | #5 | ♀ | Black (10%) |
| 10 | 7607 | 20150414 | #5 | ♂ | — |
| 11 | 7608 | 20150414 | #5-3 | ♀ | Black (10%) |
| 12 | 7609 | 20150414 | #5-3 | ♀ | — |
| 13 | 7610 | 20150414 | #5-3 | ♀ | — |
| 14 | 3 | 20150519 | #5 | Unknown | — |
| 15 | 4 | 20150519 | #5-3 | Unknown | Black (5%) |
| 16 | 5 | 20150609 | #5 | Unknown | Black (15%) |
| 17 | 6 | 20150609 | #5-3 | Unknown | Black (50%) |
| 18 | 7 | 20150616 | #4 | Unknown | Black (>90%) |

By crossing the obtained male and female chimeric chickens, it is possible to produce a ovomucoid gene-knockout homozygous chicken (G1). As the ovomucoid gene of the chicken has been knocked out, an egg laid by the chicken does not contain ovomucoid.

### Example 7: Construction of CRISPR/Cas9 Vector

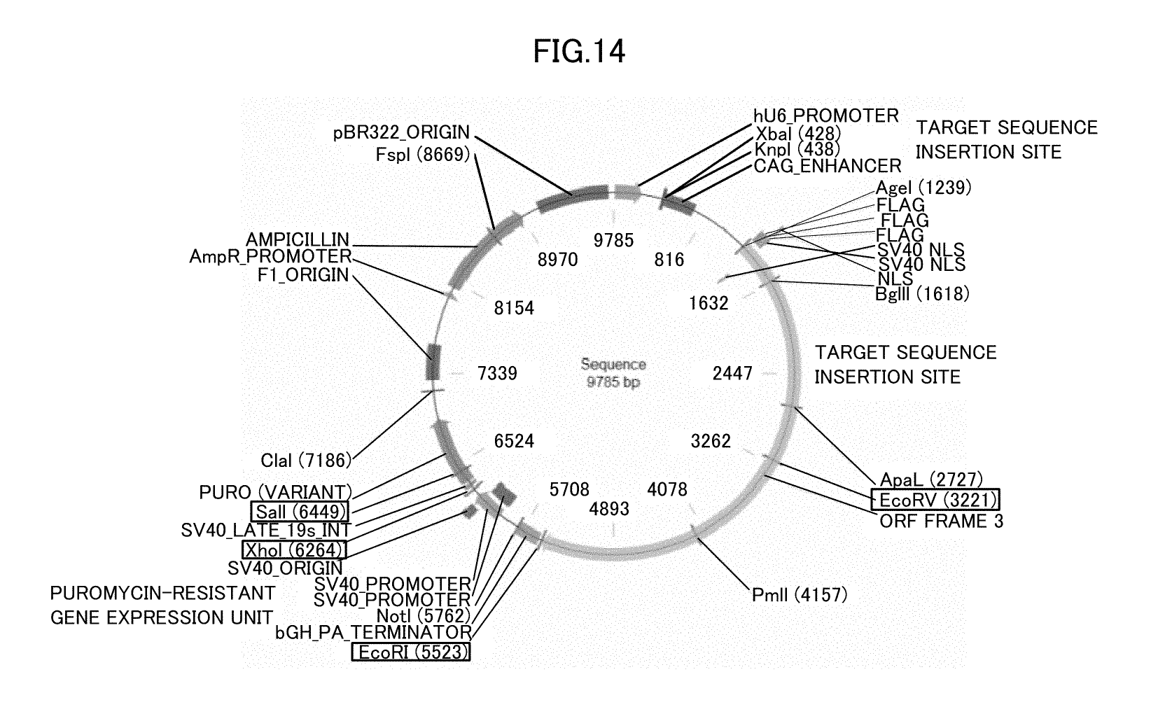

In order to construct a CRISPR/Cas9 vector, the puromycin-resistant gene was inserted into a pX330-U6-Chimeric_BB-CBh-hSpCas9 vector (manufactured by Addgene) as in the case of TALEN (see FIG. 14) in the manner described below.

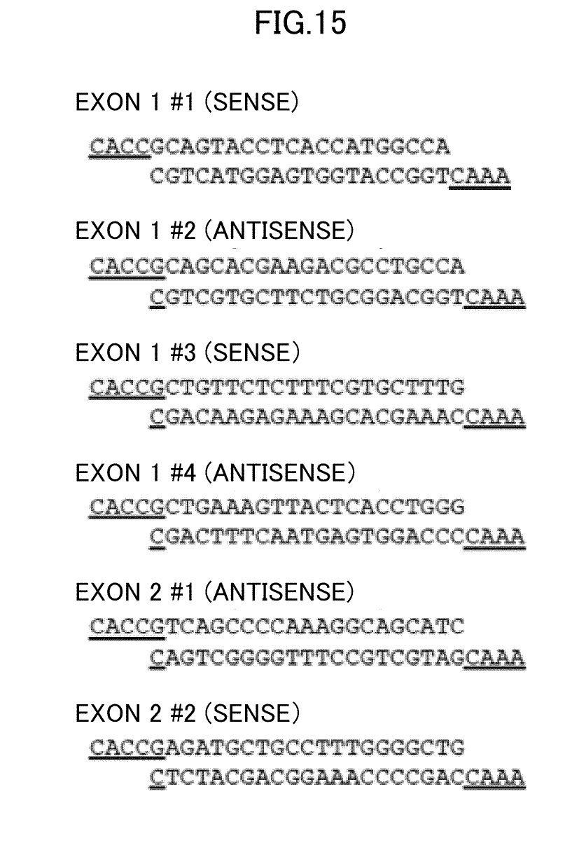

At first, target sequences capable of inducing knockout of the ovomucoid gene were searched for using “CRISPRdirect” (http://crispr.dbcls.jp/). As a result of search, target sequences at 4 sites of exon 1 and 2 sites of exon 2 were determined (SEQ ID NOS: 8 to 13). Based on the target sequences, oligo DNAs having the base sequences depicted in FIG. 15 were synthesized. In FIG. 15, “sense” targets a plus strand of the ovomucoid gene, and “antisense” targets a minus strand thereof. The underlined base sequences in FIG. 15 each represent an addition sequence to be incorporated into the vector.

Synthesized oligo DNAs were each inserted into the vector, thereby preparing 6 kinds of CRISPR/Cas9 vectors for ovomucoid knockout (CRISPR/Cas9-Pur). In addition, reporter vectors for SSA assay were prepared based on the target sequences in the manner described above, and cleavage activity of target sequence was evaluated. Regarding exon 1, the base sequences of sense oligo and antisense oligo included in the synthetic oligo that was inserted into each reporter vector for SSA assay are set forth in SEQ ID NO: 38 and SEQ ID NO: 39, respectively. Regarding exon 3, the base sequences of sense oligo and antisense oligo included in the inserted synthetic oligo are set forth in SEQ ID NO: 40 and SEQ ID NO: 41, respectively.

(Examination of Cleavage Activity of CRISPR/Cas9)

For measurement of cleavage activity of each target sequence of the CRISPR/Cas9 vector for ovomucoid knockout, SSA assay was conducted after insertion of the CRISPR/Cas9 vector into HEK293 cells. In order to test cleavage activity in chicken cells, SSA assay was conducted using epiSC in the same manner.

(Results)

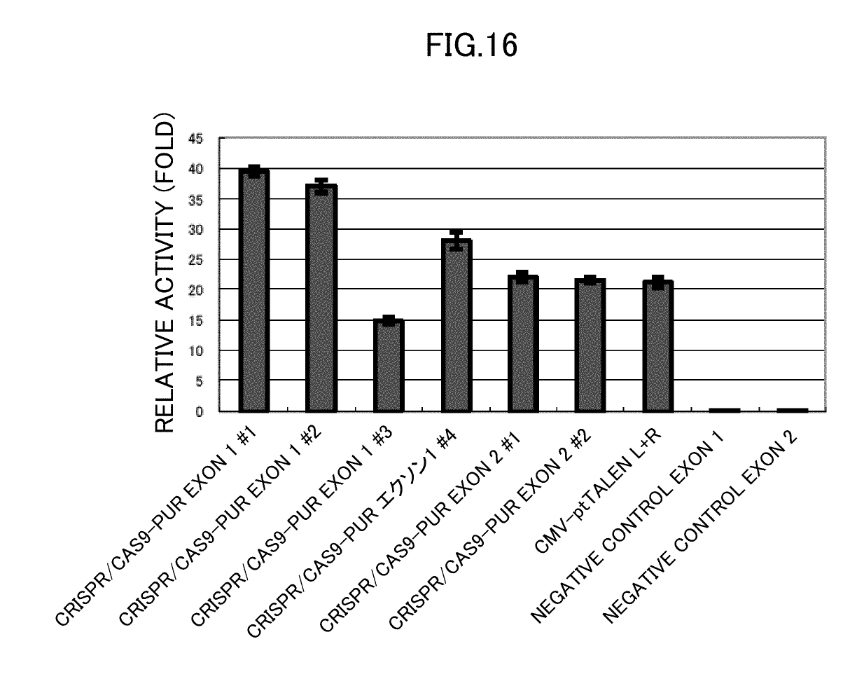

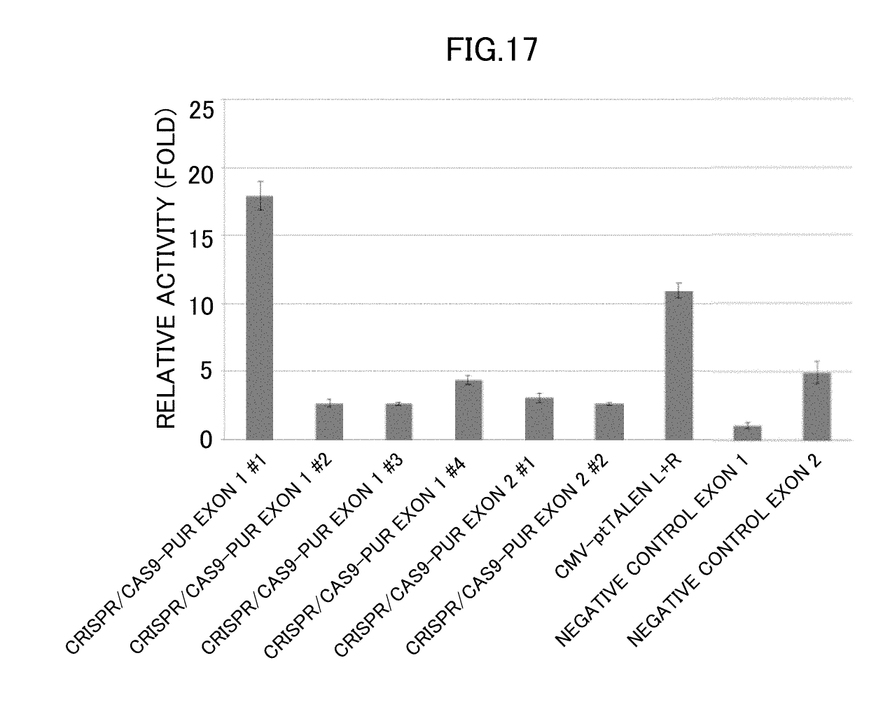

As depicted in FIG. 16, the prepared two kinds of vectors targeting exon 1 (exon 1 #1 and exon 1 #2) were found to have cleavage activity that was approximately twice higher than a high level of activity exhibited by the CMV-ptTALEN L+R vector in SSA assay. Vector #1 was found to have cleavage activity at a level comparable to that of TALEN even in epiSC as depicted in FIG. 17.

This Example suggested that the ovomucoid gene locus of chicken epiSC can be modified with the use of CRISPR/Cas9 as well. Accordingly, ovomucoid gene-knockout chickens can be produced using CRISPR/Cas9.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

This application claims the benefit of Japanese Patent Application No. 2015-168372, filed on Aug. 27, 2015, the entire disclosure of which is incorporated by reference herein.

### INDUSTRIAL APPLICABILITY

The present disclosure is desirable for production of bird eggs and particularly desirable for production of chicken eggs.

1. A bird that lays an egg that does not contain an artificially introduced foreign gene in genome thereof and has an ovomucoid content lower than that in a wild type thereof.

2. The bird according to claim 1, which includes a termination codon in at least one of first to third exons from a 5′ end of an ovomucoid gene locus, or which does not include an initiation codon in a first exon from a 5′ end of an ovomucoid gene locus.

3. The bird according to claim 1, wherein a signal sequence within the ovomucoid gene locus is modified.

4. The bird according to claim 1, which is a chicken.

5. A method for producing a bird, which comprises:
a modification step of cleaving an ovomucoid gene locus of a pluripotent stem cell of a bird with a programmable endonuclease, thereby modifying the ovomucoid gene locus; and
a transplantation step of transplanting the pluripotent stem cell containing the modified ovomucoid gene locus into an embryo of a bird.

6. The method for producing a bird according to claim 5, wherein the programmable endonuclease is transcription activator-like effector nuclease.

7. The method for producing a bird according to claim 6, wherein the transcription activator-like effector nuclease comprises a first nuclease and a second nuclease, the first nuclease comprises an amino acid sequence set forth in SEQ ID NO: 4, and the second nuclease comprises an amino acid sequence set forth in SEQ ID NO: 5.

8. The method for producing a bird according to claim 6, wherein the transcription activator-like effector nuclease comprises a third nuclease and a fourth nuclease, the third nuclease comprises an amino acid sequence set forth in SEQ ID NO: 6, and the fourth nuclease comprises an amino acid sequence set forth in SEQ ID NO: 7.

9. The method for producing a bird according to claim 5, wherein the programmable endonuclease is Cas9 nuclease in a Clustered Regularly Interspaced Short Palindromic Repeat-CRISPR associated protein system.

10. The method for producing a bird according to claim 9, wherein the Cas9 nuclease cleaves double-strand DNA in a region comprising a base sequence set forth in SEQ ID NO: 8 of the ovomucoid gene locus.

11. The method for producing a bird according to claim 9, wherein the Cas9 nuclease cleaves double-strand DNA in a region comprising a base sequence set forth in SEQ ID NO: 9 of the ovomucoid gene locus.

12. The method for producing a bird according to claim 9, wherein the Cas9 nuclease cleaves double-strand DNA in a region comprising a base sequence set forth in SEQ ID NO: 10 of the ovomucoid gene locus.

13. The method for producing a bird according to claim 9, wherein the Cas9 nuclease cleaves double-strand DNA in a region comprising a base sequence set forth in SEQ ID NO: 11 of the ovomucoid gene locus.

14. The method for producing a bird according to claim 9, wherein the Cas9 nuclease cleaves double-strand DNA in a region comprising a base sequence set forth in SEQ ID NO: 12 of the ovomucoid gene locus.

15. The method for producing a bird according to claim 9, wherein the Cas9 nuclease cleaves double-strand DNA in a region comprising base sequence set forth in SEQ ID NO: 13 of the ovomucoid gene locus.

16. The method for producing a bird according to claim 1, wherein an ovomucoid gene locus of an epiblast-derived pluripotent stem cell is modified in the modification step.

17. A bird egg that does not contain an artificially introduced foreign gene in genome thereof, and that has an ovomucoid content lower than that in a wild type thereof.


SEQUENCE LISTING (41 sequences)

SEQ ID NO: 1 - DNA, 104 bp, Gallus gallus
gagcagagcaccggcagccgcctgcagagccgggcagtacctcaccatggccatggcaggcgtcttcgtgctgttctctttcgtgctttgtggcttcctcccag

SEQ ID NO: 2 - DNA, 20 bp, Gallus gallus
atgctgcctttggggctgag

SEQ ID NO: 3 - DNA, 137 bp, Gallus gallus
gtggactgcagtaggtttcccaacgctacagacatggaaggcaaagatgtattggtttgcaacaaggacctccgccccatctgtggtaccgatggagtcacttacaccaacgattgcttgctgtgtgcctacagcgt

SEQ ID NO: 4 - protein, 964 aa, Artificial Sequence
  TALEN left for exon 1
MASSPPKKKRKVAAADYKDDDDKSWKDASGWSRMHAAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPEQVVAIASNNGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASNNGGKQALETVQRLLPVLCQDHGLTPEQVVAIASNNGGKQALETVQRLLPVLCQAHGLTPDQVVAIASHDGGKQALETVQRLLPVLCQAHGLTPAQVVAIASNNGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNNGGKQALETVQRLLPVLCQAHGLTPDQVVAIASNGGGKQALETVQRLLPVLCQAHGLTPEQVVAIASNNGGRPALESIVAQLSRPDPALAALTNDHLVALACLGGRPAMDAVKKGLPHAPELIRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 5 - protein, 998 aa, Artificial Sequence
  TALEN right for exon 1
MASSPPKKKRKVAAADYKDDDDKSWKDASGWSRMHAAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPEQVVAIASNNGGKQALETVQRLLPVLCQAHGLTPDQVVAIASNNGGKQALETVQRLLPVLCQAHGLTPAQVVAIASNIGGKQALETVQRLLPVLCQDHGLTPDQVVAIASNNGGKQALETVQRLLPVLCQDHGLTPEQVVAIASNNGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPEQVVAIASNNGGRPALESIVAQLSRPDPALAALTNDHLVALACLGGRPAMDAVKKGLPHAPELIRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 6 - protein, 998 aa, Artificial Sequence
  TALEN left for exon 3
MASSPPKKKRKVAAADYKDDDDKSWKDASGWSRMHAAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASNNGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNIGGKQALETVQRLLPVLCQDHGLTPEQVVAIASNIGGRPALESIVAQLSRPDPALAALTNDHLVALACLGGRPAMDAVKKGLPHAPELIRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 7 - protein, 964 aa, Artificial Sequence
  TALEN right for exon 3
MASSPPKKKRKVAAADYKDDDDKSWKDASGWSRMHAAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNNGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNIGGKQALETVQRLLPVLCQAHGLTPEQVVAIASNIGGRPALESIVAQLSRPDPALAALTNDHLVALACLGGRPAMDAVKKGLPHAPELIRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 8 - DNA, 20 bp, Gallus gallus
gcagtacctcaccatggcca

SEQ ID NO: 9 - DNA, 20 bp, Gallus gallus
cagcacgaagacgcctgcca

SEQ ID NO: 10 - DNA, 20 bp, Gallus gallus
ctgttctctttcgtgctttg

SEQ ID NO: 11 - DNA, 20 bp, Gallus gallus
ctgaaagttactcacctggg

SEQ ID NO: 12 - DNA, 20 bp, Gallus gallus
tcagccccaaaggcagcatc

SEQ ID NO: 13 - DNA, 20 bp, Gallus gallus
agatgctgcctttggggctg

SEQ ID NO: 14 - DNA, 20 bp, Artificial Sequence
  Oligo DNA for guide RNA, sense, exon 1 #1
gcagtacctcaccatggcca

SEQ ID NO: 15 - DNA, 20 bp, Artificial Sequence
  Oligo DNA for guide RNA, antisense, exon 1 #1
tggccatggtgaggtactgc

SEQ ID NO: 16 - DNA, 20 bp, Artificial Sequence
  Oligo DNA for guide RNA, sense, exon 1 #2
cagcacgaagacgcctgcca

SEQ ID NO: 17 - DNA, 20 bp, Artificial Sequence
  Oligo DNA for guide RNA, antisense, exon 1 #2
tggcaggcgtcttcgtgctg

SEQ ID NO: 18 - DNA, 20 bp, Artificial Sequence
  Oligo DNA for guide RNA, sense, exon 1 #3
ctgttctctttcgtgctttg

SEQ ID NO: 19 - DNA, 20 bp, Artificial Sequence
  Oligo DNA for guide RNA, antisense, exon 1 #3
caaagcacgaaagagaacag

SEQ ID NO: 20 - DNA, 20 bp, Artificial Sequence
  Oligo DNA for guide RNA, sense, exon 1 #4
ctgaaagttactcacctggg

SEQ ID NO: 21 - DNA, 20 bp, Artificial Sequence
  Oligo DNA for guide RNA, antisense, exon 1 #4
cccaggtgagtaactttcag

SEQ ID NO: 22 - DNA, 20 bp, Artificial Sequence
  Oligo DNA for guide RNA, sense, exon 2 #1
tcagccccaaaggcagcatc

SEQ ID NO: 23 - DNA, 20 bp, Artificial Sequence
  Oligo DNA for guide RNA, antisense, exon 2 #1
gatgctgcctttggggctga

SEQ ID NO: 24 - DNA, 20 bp, Artificial Sequence
  Oligo DNA for guide RNA, sense, exon 2 #2
agatgctgcctttggggctg

SEQ ID NO: 25 - DNA, 20 bp, Artificial Sequence
  Oligo DNA for guide RNA, antisense, exon 2 #2
cagccccaaaggcagcatct

SEQ ID NO: 26 - DNA, 59 bp, Artificial Sequence
  Synthetic oligonucleotide for SSA assay, exon 1-sense
gtcggatggcaggcgtcttcgtgctgttctctttcgtgctttgtggcttcctcccaggt

SEQ ID NO: 27 - DNA, 59 bp, Artificial Sequence
  Synthetic oligonucleotide for SSA assay exon 1-antisense
cggtacctgggaggaagccacaaagcacgaaagagaacagcacgaagacgcctgccatc

SEQ ID NO: 28 - DNA, 71 bp, Artificial Sequence
  Synthetic oligonucleotide for SSA assay, exon 3-sense
gtcggatgcagtaggtttcccaacgctacagacaaggaaggcaaagatgtattggtttgcaacaaggaggt

SEQ ID NO: 29 - DNA, 71 bp, Artificial Sequence
  Synthetic oligonucleotide for SSA assay, exon 3-antisense
cggtacctccttgttgcaaaccaatacatctttgccttccttgtctgtagcgttgggaaacctactgcatc

SEQ ID NO: 30 - DNA, 22 bp, Artificial Sequence
  Luc2-up-F
ctcagcaaggaggtaggtgagg

SEQ ID NO: 31 - DNA, 22 bp, Artificial Sequence
  Luc2-down-R
tgatcggtagcttcttttgcac

SEQ ID NO: 32 - DNA, 24 bp, Artificial Sequence
  Forward primer for chLIF
gcgctagccatgaggctcatcccc

SEQ ID NO: 33 - DNA, 26 bp, Artificial Sequence
  Reverse primer for chLIF
gtcgaccgcggggctgaggtgaggta

SEQ ID NO: 34 - DNA, 25 bp, Artificial Sequence
  Forward primer for genomic PCR
tgagtaacaatggaagaacactgga

SEQ ID NO: 35 - DNA, 25 bp, Artificial Sequence
  Reverse primer for genomic PCR
tgtcaggagccctaagcacaactgc

SEQ ID NO: 36 - DNA, 25 bp, Artificial Sequence
  Forward primer for genomic PCR -one vector
cctcattgtgccgctgacagattca

SEQ ID NO: 37 - DNA, 25 bp, Artificial Sequence
  Reverse primer for genomic PCR -one vector
gggagcacagaacccaacagacacc

SEQ ID NO: 38 - DNA, 111 bp, Artificial Sequence
  Synthetic oligonucleotide for SSA assay, exon 1-sense, CRISPR/Cas
gtcggatccgggcagtacctcaccatggccatggcaggcgtcttcgtgctgttctctttcgtgctttgtggcttcctcccaggtgagtaactttcagagtgctgcagaggt

SEQ ID NO: 39 - DNA, 111 bp, Artificial Sequence
  Synthetic oligonucleotide for SSA assay, exon 1-antisense, CRISPR/Cas
cggtacctctgcagcactctgaaagttactcacctgggaggaagccacaaagcacgaaagagaacagcacgaagacgcctgccatggccatggtgaggtactgcccggatc

SEQ ID NO: 40 - DNA, 51 bp, Artificial Sequence
  Synthetic oligonucleotide for SSA assay, exon 3-sense, CRISPR/Cas
gtcggatttttcccccagatgctgcctttggggctgaggtgagtaagaggt

SEQ ID NO: 41 - DNA, 51 bp, Artificial Sequence
  Synthetic oligonucleotide for SSA assay, exon 3-antisense, CRISPR/Cas
cggtacctcttactcacctcagccccaaaggcagcatctgggggaaaaatc